(19) United States
(12) Patent Application Publication
(10) Pub. No.: US 2016/0326079 A1
(43) Pub. Date: Nov. 10, 2016

(54) INTEGRATED STEAM CRACKER AND MTBE PRODUCTION UNITS

(71) Applicant: SAUDI BASIC INDUSTRIES CORPORATION, Riyadh (SA)

(72) Inventors: Guillermo Leal, Riyadh (SA); Zeeshan Nawaz, Riyadh (SA); Antonio Matarredona, Geleen (NL); Faisal Baksh, Riyadh (SA)

(21) Appl. No.: 15/109,184

(22) PCT Filed: Jan. 2, 2015
(86) PCT No.: PCT/IB2015/050029

§ 371 Date: Jun. 30, 2016

Related U.S. Application Data
(60) Provisional application No. 61/923,140, filed on Jan. 2, 2014.

(51) Int. Cl. C07C 41/06; B01J 19/24; C07C 4/04

(52) U.S. Cl. CPC C07C 41/06; C07C 4/04; B01J 19/245; B01J 2219/24

(57) ABSTRACT

Described herein are methods and systems for producing methyl t-butyl ether (MTBE) by reacting an isobutene-containing stream with methanol in an MTBE production unit. In accordance with the method described herein, at least a portion of the isobutene-containing stream is obtained from a mixed C4 stream from a steam cracking unit and is not produced by passing the dehydrogenation of isobutane. Furthermore, an alkane-containing stream from the MTBE production unit is used as a feed stream for the steam cracking unit.

### TECHNICAL FIELD

The invention concerns methods and systems for increasing the efficiency of a methyl tert-butyl ether production unit. More particularly, the invention concerns methods and systems for integrating a steam cracker unit with a methyl tert-butyl ether production unit by redirecting product streams between the units.

### BACKGROUND

Methyl tert-butyl ether (MTBE) is an aliphatic alkyl ether that is used as a gasoline additive to increase the octane rating of gasoline products. Typically, MTBE is produced on a large scale by reaction of isobutene with methanol according to reaction (I)

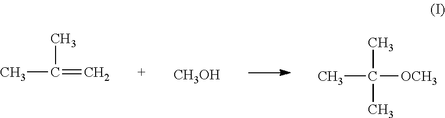

The conventional synthesis of MTBE may be accomplished in many different ways, but one typical commercial process for the manufacture of MTBE is based upon a liquid phase reaction of isobutene and methanol catalyzed by cationic ion-exchange resin (see, e.g., Izquierdo, J. F., Cunill, F., Vila M., Tejero J. and Tborra M. Equilibrium constants for methyl tertiary butyl ether liquid-phase synthesis. Journal of Chemical and Engineering Data, 1992, vol. 37, p. 339; Brockwell, H. L., Sarathy P. R. and Trotta R. Synthesize ethers. Hydrocarbon Processing, 1991, vol. 70, No. 9, p. 133; Chemical Economics Handbook, Gasoline Octane Improvers. CEH Marketing Report, 1986, p. 543, Stanford Research Institute, SRI International, Menlo Park, Calif.).

Conventionally, the isobutene for the reaction is produced by dehydrogenation of isobutane. However, this dehydrogenation step is reversible, endothermic, and accompanied by a volume expansion. Accordingly, it is advantageous to drive the dehydrogenation reactions by running it at higher temperatures and lower pressures. The problem with this approach, however, is that high reaction temperatures promote unwanted side reactions, including coke formation and catalyst deactivation. In this way, the dehydrogenation reaction can be a bottleneck in the process for producing MTBE, and it can result in MTBE production units running at less than full capacity.

Accordingly, in view of increased demand for MTBE, methods are sought to increase the efficiency of the process.

### SUMMARY

One aspect of the invention provides a method for producing methyl-t-butyl ether. The method comprises reacting an isobutene-containing stream with methanol in a methyl tert-butyl ether production unit, where the isobutene-containing stream is a mixed C4 product stream from a steam cracking unit and is not produced via dehydrogenation of isobutene. The method also include the step of sending an alkane stream from an isomerization unit of the methyl tert-butyl ether production unit to the steam cracking unit to serve as an feed stream for the steam cracking unit.

Another aspect of the invention is to provide a system for producing methyl-t-butyl ether. The system includes a methyl tert-butyl ether production unit and a steam cracking unit. An isobutene-containing stream that is a mixed C4 product stream from the steam cracking unit and which is not produced via dehydrogenation of isobutene is used as a feed stream for the methyl tert-butyl ether production unit. One or more alkane streams from an isomerization unit of the methyl tert-butyl ether production unit is sent to the steam cracking unit to serve as a feed stream for the steam cracking unit.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 shows a schematic drawing of a process flow according to one implementation of the invention.

FIG. 2 shows a schematic drawing of a process flow according to one implementation of the invention.

FIG. 3 is a schematic process diagram showing exemplary methods and systems of integration of a steam cracker unit (top half of diagram) with an MTBE unit (bottom half of diagram). Possible integrating features include any or all of the following, as illustrated: Case A: A butadiene extraction unit in the steam cracker unit, which may be further attached to an n-butane fractionator for separating n-butane from the butene stream; Case B: Swapping pure n-butane from the MTBE-BIC[DIB] column to the steam cracker, in place of (or in addition to) a recycled hydrogenated butene stream; Case C: Swapping mixed butanes (primarily isobutane) from the MTBE recycling stream as feed to the cracker; Case D: Isobutene-rich stream swapping (without butadiene extraction) to the MTBE synthesis unit; Case E: Swapping mixed butane from the refinery (comprising about 70-80% n-butane) to the cracker as feed; Case F: Isobutene-rich stream swapping (following butadiene and/or n-butane extraction) to the MTBE synthesis unit.

### DETAILED DESCRIPTION

The invention concerns methods and systems for increasing the efficiency of an MTBE production unit. One aspect of the invention is the recognition that it is advantageous to use an isobutene-containing product stream from a steam cracking unit as a feed stream for an MTBE production unit, and to use an alkane stream from the MTBE production unit (e.g., from the isomerization unit of the MTBE production unit) as a feed stream for the steam cracking unit. By redirecting product streams between the units as described herein, significant savings in cost and improved efficiency can be achieved.

The present invention provides, among other things, an improved method for producing methyl-t-butyl ether (MTBE). The method comprises reacting an isobutene-containing stream with methanol in an MTBE production unit, where the isobutene-containing stream is a mixed C4 product stream from a steam cracking unit (e.g., a distillate cut obtained by distillation of C4 crudes) and is not produced via dehydrogenation of isobutene in a separate dehydrogenation unit. In certain embodiments, the isobutene that is used to produce MTBE is obtained solely from a product stream of the steam cracking unit. In other embodiments, the isobutene-containing product stream from the steam cracking unit is combined with isobutene that is produced from a conventional dehydrogenation unit that converts isobutane to isobutene. In this way, MTBE production may be increased by supplementing the isobutene feed, normally produced wholly by dehydrogenation of isobutane, with isobutene recovered from the C4 fraction of the steam cracker. As described herein, some or all of the unreacted components of this C4 stream, following MTBE production, can be recycled back to the steam cracker. In one non-limiting example, the C4 stream from the steam cracking system may be treated to remove butadiene and/or n-butane, typically in separate operations, prior to reaction with methanol. The removed n-butane may then be redirected as feed to the steam cracking system.

The improved method of producing MTBE also includes the step of sending an alkane stream from an isomerization unit of the MTBE production unit to the steam cracking unit to serve as a feed stream for the steam cracking unit. If desired, at least a portion of a stream of mixed butanes influent to the isobutane/n-butane separation unit of the MTBE system is diverted as feed to the steam cracking system. For example, the MTBE production unit may be configured to include an isobutane/n-butane separation unit, and at least a portion of a stream of n-butane produced by the isobutane/n-butane separation unit of the MTBE system may be directed as feed to the steam cracking system. Such diversion serves to compensate at least in part for the isobutene-containing stream from the steam cracking unit that is used as a feed stream for the MTBE reaction. If desired, a recycle stream containing isobutane and/or n-butane, produced after separation of MTBE from the product of reaction of the isobutene-containing stream with methanol, also (or alternatively) may be directed as feed to the steam cracking system. This recycle stream may be hydrogenated prior to being directed as feed to the steam cracking system. 1-Butene may be removed from the recycle stream prior to its being directed as feed to the steam cracking system.

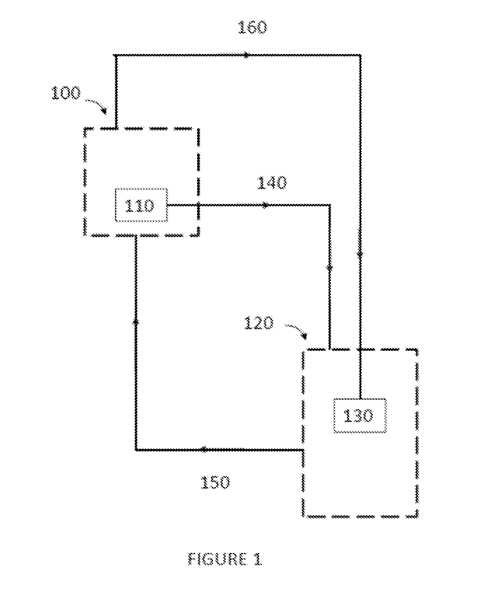

FIG. 1 shows a schematic drawing that illustrates one implementation of the invention. FIG. 1 shows MTBE production unit 100, which includes isomerization unit 110, and steam cracking unit 120, which includes hydrogenation unit 130. As the skilled artisan will appreciate, the process that is illustrated in FIG. 1 is a highly simplified schematic and MTBE production unit 100 and steam cracking unit 120 typically will contain many more components than those illustrated in FIG. 1. Product stream 150 from steam cracking unit 120 is a distillate cut that contains isobutene (and other C4 molecules, such as isobutane) and is used as a feed stream for MTBE production unit 100. As noted previously, isobutene-containing feed stream 150 may be the sole source of isobutene for the MTBE production unit 100, which reacts isobutene with methanol to form MTBE. Alternatively, isobutene-containing feed stream 150 may be combined with or used in conjunction with an isobutene—stream obtained by conventional dehydrogenation of isobutane to produce MTBE. To compensate for using the isobutene-containing stream as a feed stream for MTBE production unit 100, one or more alkane-containing streams from MTBE production unit 100 may be directed to steam cracking unit 120. For instance, an n-butane containing alkane stream 140 may be directed to steam cracking unit 120 from isomerization unit 110 of MTBE production unit 100. In addition (or in the alternative), product stream 160, which is produced after the isobutene in isobutene-containing feed stream 150 reacts with methanol to form MTBE, may be sent to steam cracking unit 120. Compared to the case for product stream 150 from steam cracking unit 120, the relative amount of isobutane to isobutene in product stream 160 is higher, because isobutene that was in product stream 150 is consumed during MTBE production. In certain embodiments, it is preferable to direct an n-butane containing stream (e.g., n-butane containing alkane stream 140), rather than an isobutane/isobutene-containing stream (e.g., product stream 160) because n-butane produces around 6-7 times more ethylene (in terms of weight percent) when recycled to the steam cracking unit 120.

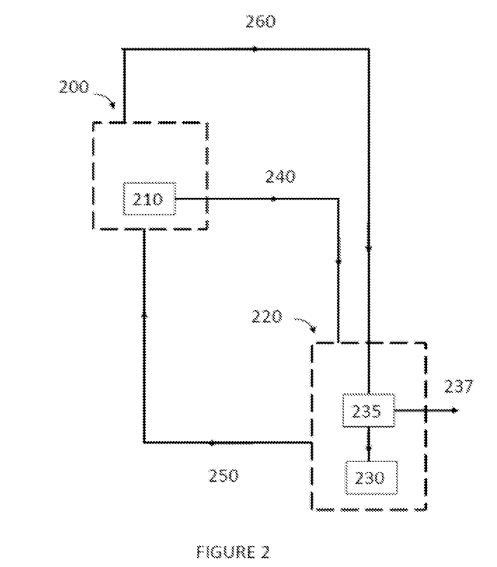

FIG. 2 shows a schematic drawing that illustrates another implementation of the invention. FIG. 2 shows MTBE production unit 200, which includes isomerization unit 210, and steam cracking unit 220, which includes hydrogenation unit 230. Like FIG. 1, the process that is illustrated in FIG. 2 is a highly simplified schematic, and the skilled artisan will recognize that MTBE production unit 200 and steam cracking unit 220 typically will contain many more components than those illustrated in FIG. 2. Product stream 250 from steam cracking unit 220 is a distillate cut that contains isobutene (and other C4 molecules, such as isobutane) and is used as a feed stream for MTBE production unit 200. The steam cracking unit 220 in FIG. 2 also features separation unit 235 which separates out 1-butene or butadiene (represented as stream 237) before hydrogenation and before the product stream identified as isobutene-feed stream 250 is sent to MTBE production unit 200. By doing so, the volume of feedstock to MTBE production unit 200 is advantageously reduced. Similar to the embodiment illustrated in FIG. 1, isobutene-containing feed stream 250 may be the sole source of isobutene for the MTBE production unit 200 or may be combined with or used in conjunction with an isobutene-stream obtained by conventional dehydrogenation of isobutane to produce MTBE. To compensate for using the isobutene-containing stream as a feed stream for MTBE production unit 200, one or more alkane-containing streams from MTBE production unit 200 may be directed to steam cracking unit 220. For instance, an n-butane containing alkane stream 240 may be directed to steam cracking unit 220 from isomerization unit 210 of MTBE production unit 200. In addition (or in the alternative), product stream 260, which is produced after the isobutene in isobutene-containing feed stream 250 reacts with methanol to form MTBE, may be sent to steam cracking unit 220. Compared to the case for product stream 250 from steam cracking unit 220, the relative amount of isobutane to isobutene in product stream 260 is higher, because the isobutene in product stream 250 is consumed during MTBE production.

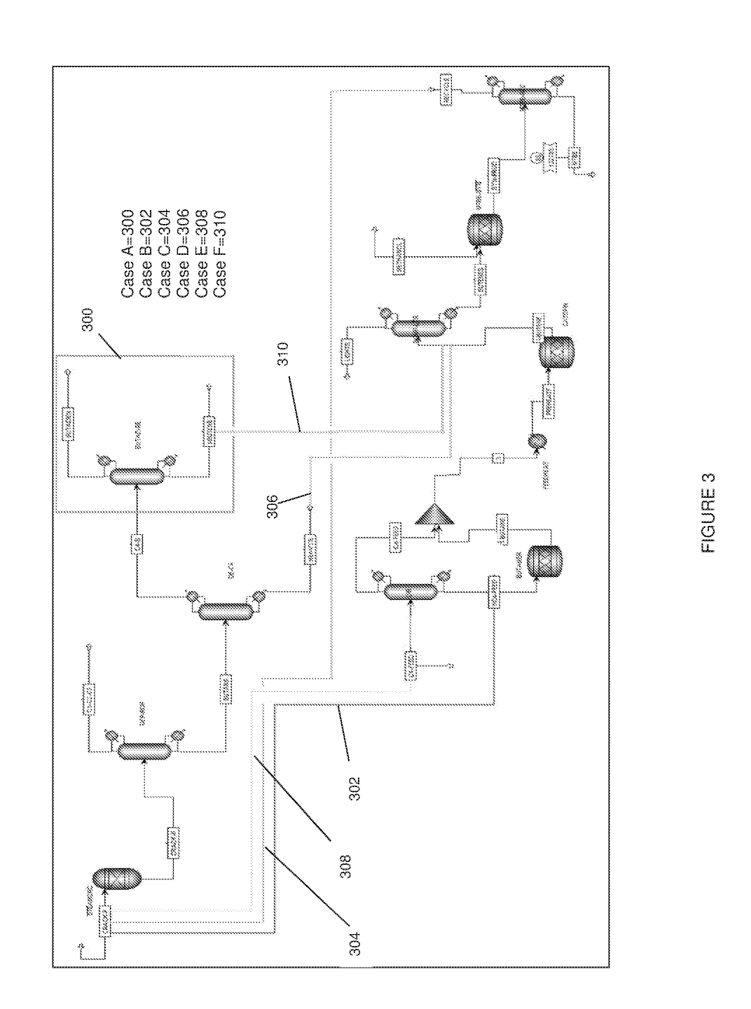

FIG. 3 shows various possible systems contemplated by the invention for improving the efficiency of MTBE production. FIG. 3 is a schematic process diagram showing exemplary methods and systems of integration of a steam cracker unit (top half of diagram) with an MTBE unit (bottom half of diagram). Possible integrating features include any or all of the following, as illustrated: Case A 300: A butadiene extraction unit in the steam cracker unit, which may be further attached to an n-butane fractionator for separating n-butane from the butene stream; Case B 302: Swapping pure n-butane from the MTBE-BIC[DIB] column to the steam cracker, in place of (or in addition to) a recycled hydrogenated butene stream; Case C 304: Swapping mixed butanes (primarily isobutane) from the MTBE recycling stream as feed to the cracker; Case D 306: Isobutene-rich stream swapping (without butadiene extraction) to the MTBE synthesis unit; Case E 308: Swapping mixed butane from the refinery (comprising about 70-80% n-butane) to the cracker as feed; Case F 310: Isobutene-rich stream swapping (following butadiene and/or n-butane extraction) to the MTBE synthesis unit.As discussed herein, these systems involve “stream swapping” to provide an isobutene to an MTBE unit, by diverting an isobutene-rich stream from a steam cracker processing unit. See, for example, the arrows labeled “Case D” 306 and “Case F” 310 in FIG. 3. This stream may be produced by distillation of the crude C4 fraction from the steam crackers and contains isobutane/isobutene in addition to other components, notably butadiene, 1-butene and n butane. The stream may be sent to a butadiene or n-butane unit for removal of same (“Case A” 300 in FIG. 3) prior to being sent to the MTBE unit. This is advantageous because the volume as feedstock to the MTBE unit is considerably reduced. In addition, a separate source of butadiene is obtained. The n-butane separated can be recycled to a steam cracker unit, as indicated in FIG. 3 (“Case A” 300). The remaining isobutene/isobutane-rich stream may typically comprise about 2:1 isobutene:isobutane.

The isobutene in the isobutene/isobutane-rich stream then reacts with methanol in the MTBE production unit, leaving an isobutane-rich stream for other purposes. In a preferred embodiment, the isobutane-rich stream is recycled as feedstock for the steam cracker (see arrow labeled “Case C” 304 in FIG. 3). 1-Butene may be recovered from this recycle stream (see “Butene-1”, right side of FIG. 3) prior to recycling the remaining mixed butanes to the steam crackers. Alternatively, this stream can be sent as part of the MTBE recycle to the dehydrogenation reactor in the MTBE unit. MTBE production may be increased by supplementing the isobutene feed, normally produced wholly by dehydrogenation of isobutane, with isobutene recovered from the C4 fraction of the steam cracker. As stated above, some or all of the unreacted components of this C4 stream, following MTBE production, can be recycled back to the steam cracker.

To compensate further for the diversion of the isobutane/isobutene-containing stream from the steam cracker distillation columns, an n-butane stream can be diverted from the isomerization unit in the MTBE process to a steam cracker (see arrow labeled “Case B” in FIG. 3). This will not only enhance ethylene production from the cracker but can also reduce or eliminate a hydrogenation section from the cracker setup.

Mixed C4's from the refinery, containing about 70-80% n butane, may also be diverted to the steam crackers (see arrow labeled “Case E” in FIG. 3). The resulting increased feed of n butane to the steam crackers is expected to increase yields of ethylene and propylene in the steam cracking process, a further advantage of the integrated process. For example, cracking n-butane at a conversion of 96% per pass typically yields about 40% ethylene and 14% propylene. An increased feed of isobutane is also expected to increase yields of ethylene and propylene, though to a lesser degree than n-butane.

Accordingly, MTBE production in an MTBE unit is increased by supplementing the isobutene feed with an isobutene-rich stream recovered from the C4 fraction of a steam cracker, as described above; and C4 streams from the MTBE unit, typically from the isomerization unit, can be diverted to the steam cracker, as described above, to replace the diverted isobutene-rich stream from the steam cracker.

The invention also recognizes that the financial impact of reconfiguring product streams as discussed herein may be determined using computer simulation techniques, non-limiting examples of which include linear programming (LP) and SPYRO® (Technip Benelux B.V., the Netherlands). In particular, for a given petrochemical refining plant, it is advantageous to use such computer simulation techniques prior to making any capital expenditures to assess the financial impact of reconfiguring product streams. These computer simulation techniques also may be used to determine whether it is financially feasible to add additional units (e.g., a butene/butadiene separation unit) or remove certain units (e.g., hydrogenation units).

The MTBE production unit and the steam cracking unit may or may not be in physical proximity to each other. In certain preferred embodiments, the MTBE production unit and the steam cracking unit are located at the same petrochemical refining site. However, the invention also explicitly contemplates situations in which the MTBE production unit and the steam cracking unit are located at different sites. Preferably, the MTBE production unit and the steam cracking unit are fluidly connected, although it is not required.

The steam cracking process that is contemplated by the present invention is not particularly limited and may be performed in accordance with any known steam cracking process used in the petrochemical arts. Generally, steam cracking is a process by which saturated hydrocarbons are broken down into smaller, often unsaturated, hydrocarbons. Among other things, the steam used in the steam cracking reaction enables the catalyst particles to be maintained in a fluidized state. When contacting with the fluidized catalyst in a reactor, the hydrocarbon feedstock and steam may be introduced either in co-current or counter-current manner, with the fluidized catalyst in arranged such that hydrocarbon feedstock is introduced into the catalyst under force of gravity.

The temperature of steam introduced into the catalyst bed can 750-1100° C., more preferably 800-1000° C. It is often desirable to maintain the catalyst bed in a fluid state at a temperature of 600-800° C. The hydrocarbon feedstock preferably has a temperature below 400° C., below 300° C., or below 250° C. In certain embodiments, the hydrocarbon feedstock has a temperature of 200-300° C. at the point of entry into the reactor. As will be appreciated by those of skill in the art, the temperature of the feedstock at its point of entry into the steam cracking unit may be controlled either by adjusting the position of an injection nozzle relative to the catalyst bed or by adjusting a stem annulus surrounding the nozzle in order to decrease the hydrocarbon feedstock temperature.

Steam cracking results in the conversion of heavier materials into lower molecular weight products can be separated into streams of similar sized hydrocarbons. For instance, steam cracking many be used to produce a C4 stream containing a mixture of difference C4 species, including n-butane, isobutane, and isomeric butenes (e.g. 1-butene, cis-and trans-2-butene, and isobutene), and 1,3-butadiene. In addition, such C4 streams may contain one or more other chemical species, non-limiting examples of which include ethyl acetylene, dimethyl acetylene, vinyl acetylene, and diacetylene. The products obtained depend on the composition of the feed, the hydrocarbon-to-steam ratio, and on the cracking temperature and furnace residence time.

The chemical reaction used to produce MTBE is not particularly limited, and can be any reaction that is compatible with the isobutene-containing feedstream from the steam cracker unit. In certain preferred embodiments, the chemical reaction used to produce MTBE is a liquid phase reaction of isobutene and methanol catalyzed by cationic ion-exchange resin (see, e.g., Izquierdo, J. F., Cunill, F., Vila M., Tejero J. and Tborra M. Equilibrium constants for methyl tertiary butyl ether liquid-phase synthesis. Journal of Chemical and Engineering Data, 1992, vol. 37, p. 339.; Brockwell, H. L., Sarathy P. R. and Trotta R. Synthesize ethers. Hydrocarbon Processing, 1991, vol. 70, No. 9, p. 133.; Chemical Economics Handbook, Gasoline Octane Improvers. CEH Marketing Report, 1986, p. 543, Stanford Research Institute, SRI International, Menlo Park, Calif.).

The method and system for producing methyl tert-butyl ether (MTBE) includes at least the following embodiments:

Embodiment 1: A method for producing methyl tert-butyl ether, comprising: reacting an isobutene-containing stream with methanol to form methyl tert-butyl ether in a methyl tert-butyl ether production unit, wherein the isobutene-containing stream comprises a mixed C4 product stream from a steam cracking unit; and sending an alkane stream from an isomerization unit of the methyl tert-butyl ether production unit to the steam cracking unit to serve as a feed stream for the steam cracking unit.

Embodiment 2: The method of claim 1, wherein the alkane stream contains predominantly a C4 alkane selected from the group consisting of n-butane and isobutane.

Embodiment 3: The method of claim 1 or claim 2, wherein the predominant C4 alkane in the alkane stream is n-butane.

Embodiment 4: The method of claim 1 or claim 2, wherein the predominant C4 alkane in the alkane stream is isobutane.

Embodiment 5: The method of any of claims 1-4, wherein the alkane stream is treated to remove 1-butene prior to passing through a hydrogenation unit of the steam cracking unit.

Embodiment 6: The method of any of claims 1-5, wherein the mixed C4 stream from the steam cracking unit is treated to remove butadiene prior to reaction with methanol.

Embodiment 7: The method of any of claims 1 to 6, wherein the mixed C4 stream from the steam cracking unit is treated to remove n-butane prior to reaction with methanol.

Embodiment 8: The method of claim 7, wherein said removed n-butane is directed as feed to said steam cracking system.

Embodiment 9: The method of any of claims 1-9, further comprising producing ethylene from cracking of alkanes in said steam cracking system.

Embodiment 10: A system for producing methyl-t-butyl ether, comprising: a methyl tert-butyl ether production unit; and a steam cracking unit; wherein an isobutene-containing stream comprising a mixed C4 product stream from the steam cracking unit and which is not produced via dehydrogenation of isobutene is used as a feed stream for the methyl tert-butyl ether production unit; and wherein one or more alkane streams from an isomerization unit of the methyl tert-butyl ether production unit is sent to the steam cracking unit to serve as a feed stream for the steam cracking unit.

Embodiment 11: The system of claim 10, wherein the alkane stream contains predominantly a C4 alkane selected from the group consisting of n-butane and isobutane.

Embodiment 12: The system of claim 10 or claim 11, wherein the predominant C4 alkane in the alkane stream is n-butane.

Embodiment 13: The system of claim 10 or claim 11, wherein the predominant C4 alkane in the alkane stream is isobutane.

Embodiment 14: The system of any of claims 10 to 13, wherein the alkane stream is treated to remove 1-butene prior to passing through a hydrogenation unit of the steam cracking unit.

Embodiment 15: The system of any of claims 10-14, wherein the mixed C4 stream from the steam cracking unit is treated to remove butadiene prior to reaction with methanol.

Embodiment 16: The system of any of claims 10-14, wherein the mixed C4 stream from the steam cracking unit is treated to remove n-butane prior to reaction with methanol.

Embodiment 17: The system of claim 16, wherein said removed n-butane is directed as feed to said steam cracking system.

These and other applications and implementations will be apparent in view of the disclosure. Such modifications, substitutions and alternatives can be made without departing from the spirit and scope of the invention, which should be determined from the appended claims. While the present device, system, and method have been described with reference to several embodiments and uses, and several drawings, it will be appreciated that features and variations illustrated or described with respect to different embodiments, uses, and drawings can be combined in a single embodiment.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

I/we claim:

1. A method for producing methyl tert-butyl ether, comprising:
reacting an isobutene-containing stream with methanol to form methyl tert-butyl ether in a methyl tert-butyl ether production unit, wherein the isobutene-containing stream comprises a mixed C4 product stream from a steam cracking unit; and
sending an alkane stream from an isomerization unit of the methyl tert-butyl ether production unit to the steam cracking unit to serve as a feed stream for the steam cracking unit.

2. The method of claim 1, wherein the alkane stream contains predominantly a C4 alkane selected from the group consisting of n-butane and isobutane.

3. The method of claim 1, wherein the predominant C4 alkane in the alkane stream is n-butane.

4. The method of claim 1, wherein the predominant C4 alkane in the alkane stream is isobutane.

5. The method of claim 1, wherein the alkane stream is treated to remove 1-butene prior to passing through a hydrogenation unit of the steam cracking unit.

6. The method of claim 1, wherein the mixed C4 stream from the steam cracking unit is treated to remove butadiene prior to reaction with methanol.

7. The method of claim 1, wherein the mixed C4 stream from the steam cracking unit is treated to remove n-butane prior to reaction with methanol.

8. The method of claim 7, wherein said removed n-butane is directed as feed to said steam cracking system.

9. The method of claim 1, further comprising producing ethylene from cracking of alkanes in said steam cracking system.

10. A system for producing methyl-t-butyl ether, comprising:
a methyl tert-butyl ether production unit; and
a steam cracking unit;
wherein an isobutene-containing stream comprising a mixed C4 product stream from the steam cracking unit and which is not produced via dehydrogenation of isobutene is used as a feed stream for the methyl tert-butyl ether production unit; and
wherein one or more alkane streams from an isomerization unit of the methyl tert-butyl ether production unit is sent to the steam cracking unit to serve as a feed stream for the steam cracking unit.

11. The system of claim 10, wherein the alkane stream contains predominantly a C4 alkane selected from the group consisting of n-butane and isobutane.

12. The system of claim 10, wherein the predominant C4 alkane in the alkane stream is n-butane.

13. The system of claim 10, wherein the predominant C4 alkane in the alkane stream is isobutane.

14. The system of claim 10, wherein the alkane stream is treated to remove 1-butene prior to passing through a hydrogenation unit of the steam cracking unit.

15. The system of claim 10, wherein the mixed C4 stream from the steam cracking unit is treated to remove butadiene prior to reaction with methanol.

16. The system of claim 10, wherein the mixed C4 stream from the steam cracking unit is treated to remove n-butane prior to reaction with methanol.

17. The system of claim 16, wherein said removed n-butane is directed as feed to said steam cracking system.
